(19) United States
(12) Patent Application Publication
(10) Pub. No.: US 2023/0065206 A1
(43) Pub. Date: Mar. 2, 2023

(54) SYSTEM AND METHOD FOR REPAIRING A LIGAMENT

(71) Applicant: Pedro Juan LOREDO, Southlake, TX (US)

(72) Inventor: Pedro Juan LOREDO, Southlake, TX (US)

(21) Appl. No.: 17/411,150

(22) Filed: Aug. 25, 2021

(51) Int. Cl. A61F 2/08

(52) U.S. Cl. CPC A61F 2/0805; A61F 2/0811; A61F 2002/0888; A61F 2210/0004; A61F 2220/0075

(57) ABSTRACT

A surgical kit for repairing a ligament includes suture tape configured to be positioned across the ligament from a fixed end of the ligament to a free end of the ligament. At least one anchor is configured to position over the ligament. An instrument is configure to secure the anchor to the bone.

### TECHNICAL FIELD

The present invention is directed to a system and method for repairing a ligament and, more particularly, to a system and method of repairing a ligament that does not require drilling into the patient's bone.

### BACKGROUND

Generally, during an orthopaedic surgery for repairing a ligament, the torn ligament is stretched over the respective joint and secured at both ends of the joint. That is, the ligament is stretched from a bone to an adjacent bone over the joint. The ligament is then held in place by suture tape positioned over the ligament. To secure the suture tape, an anchor is inserted into each of the bones. The anchor is inserted after pre-drilling into the respective bone. The suture tape is secured to each anchor.

Notably, this method requires drilling into the bone, which can be time consuming and extend the overall time that the patient is required to be anesthetized. Drilling into the bone may also cause structural damage to the bone if not done correctly. If the anchor is not properly set within the bone, the patient may require follow up surgeries and may experience permanent damage to the bone.

### SUMMARY

The present disclosure includes one or more of the features recited in the appended claims and/or the following features which, alone or in any combination, may comprise patentable subject matter.

According to a first aspect of the disclosed embodiments, a method of repairing a ligament is provided. The ligament includes a fixed end fixed to a first bone and a free end disconnected from an adjacent second bone. The method includes extending the free end of the ligament over a joint between the first bone and the second bone. The method also includes positioning the free end of the ligament on the second bone. The method also includes positioning suture tape across the ligament from the fixed end of the ligament to the free end of the ligament. The method also includes positioning a first anchor over the fixed end of the ligament. The method also includes securing the first anchor to the first bone by pressing a tine of the first anchor into the first bone. A first end of the suture tape and the fixed end of the ligament are secured between the first bone and the first anchor. The method also includes positioning a second anchor over the free end of the ligament. The method also includes securing the second anchor to the second bone by pressing a tine of the second anchor into the second bone. A second end of the suture tape and the free end of the ligament are secured between the second bone and the second anchor.

In some embodiments of the first aspect, the method may include securing the first anchor to the first bone by squeezing the first anchor with a clamp, and securing the second anchor to the second bone by squeezing the second anchor with the clamp. The method may include applying a pressure to the respective anchor with a first arm of the clamp, and applying the pressure to the respective bone with a second arm of the clamp. The method may include securing the first anchor to the first bone by applying force to the first anchor with a mallet, and securing the second anchor to the second bone by applying force to the second anchor with the mallet. The method may include removeably securing each respective anchor to an end of a pin, and applying the respective force to the pin with the mallet. The method may include extending the free end of the ligament over the joint to a distance that achieves a predetermined tension in the ligament. Each respective anchor may include a plurality of tines. The method may include pressing each of the plurality of tines into the respective bone. The ligament may be repaired at a surgical site. The method may include closing the surgical site with the suture tape, the first anchor, and the second anchor positioned within the surgical site. Positioning the suture tape across the ligament may include positioning a suture tape that includes an absorbable material. The method may include repairing the ligament without drilling into either the first bone or the second bone.

According to a first aspect of the disclosed embodiments, a surgical kit for repairing a ligament is provided. The ligament includes a fixed end fixed to a first bone and a free end disconnected from an adjacent second bone. The kit includes suture tape configured to be positioned across the ligament from the fixed end of the ligament to the free end of the ligament. A first anchor is configured to position over the fixed end of the ligament. A second anchor is configured to be positioned over the free end of the ligament. An instrument is configured to secure the first anchor to the first bone by pressing a tine of the first anchor into the first bone. A first end of the suture tape and the fixed end of the ligament are configured to be secured between the first bone and the first anchor. The instrument is further configured to secure the second anchor to the second bone by pressing a tine of the second anchor into the second bone. A second end of the suture tape and the free end of the ligament are configured to be secured between the second bone and the second anchor.

In some embodiments of the second aspect, the instrument may include a clamp. The clamp may include a first arm configured to apply pressure to the respective anchor, and a second arm configured to apply pressure to the respective bone. Each respective anchor may be removeably secured to the first arm of the clamp. The instrument may include a mallet. A pin may be configured to position against the respective anchor. Force may be applied to the pin with the mallet. Each respective anchor may be removeably secured to the pin. Each respective anchor may include a plurality of tines. Each respective anchor may include a first tine configured to position on a first side of the suture tape, and a second tine configured to position on a second side of the suture tape. The suture tape may include an absorbable material.

Additional features, which alone or in combination with any other feature(s), such as those listed above and/or those listed in the claims, can comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 is a top view of a roll of suture tape configured to be included in a kit for repairing a ligament;

FIG. 2 is a perspective view of an anchor configured to be included in the kit;

FIG. 3 is a perspective view of an instrument configured to be included in the kit;

FIG. 4 is a perspective view of an arm of the instrument shown in FIG. 3;

FIG. 5 is a perspective view of the anchor shown in FIG. 2 removeably coupled to the arm of the instrument shown in FIG. 4;

FIG. 6 is a side view of another instrument configured to be included in the kit;

FIG. 7 is a side view of a pin configured to be used with the instrument shown in FIG. 6;

FIG. 8 is an end view of the pin shown in FIG. 7;

FIG. 9 is a perspective view of the anchor shown in FIG. 2 removeably coupled to the end of the pin shown in FIG. 7;

FIG. 10 is a perspective view of a damaged ligament in a patient's hand;

FIG. 11 is a perspective view of the damaged ligament shown in FIG. 10 extended over a joint to a repaired position;

FIG. 12 is a perspective view of a pair of anchors shown in FIG. 2 securing the suture tape shown in FIG. 1 over the repaired ligament;

FIG. 13 is a perspective view of the instrument shown in FIG. 3 utilized to secure an anchor to the patient's bone; and

FIG. 14 is a perspective view of the instrument shown in FIG. 6 and the pin shown in FIG. 7 utilized to secure an anchor to the patient's bone.

### DETAILED DESCRIPTION

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

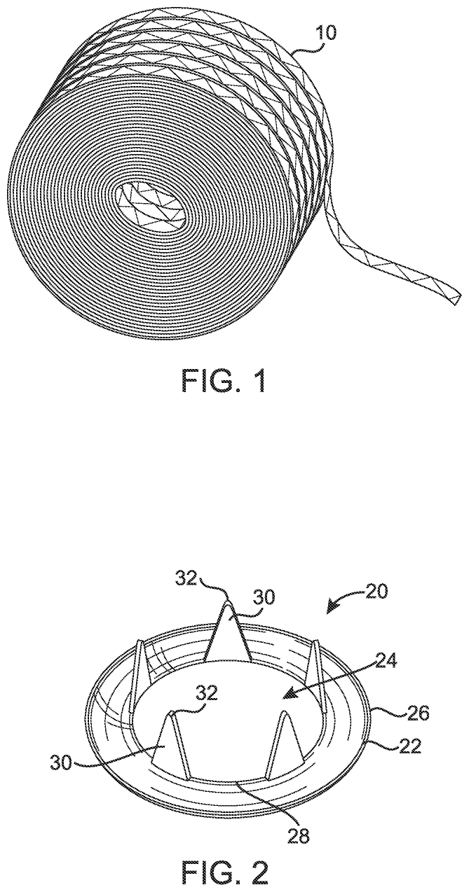

Referring to FIG. 1, a kit for repairing a ligament at a surgical site includes suture tape 10. The suture tape 10 is configured to increase a suture footprint allowing for increased soft tissue-to-bone contact in soft tissue repairs, e.g. ligament repairs. The suture tape 10 holds the ligament in place post-surgery to allow the ligament to reconnect to the bone. The suture tape 10 may be formed from an absorbable material that is absorbed into the patient's body over time post-surgery.

Referring now to FIG. 2, the kit also includes a plurality of anchors 20 that are configured to retain the suture tape 10 in position post-surgery. The method described below teaches the use of two anchors 20 to retain the suture tape 10; however, it will be noted that any number of anchors 20 may be utilized during surgery. Accordingly, the kit may provide any number of anchors 20.

Each anchor 20 includes a base 22 that is illustrated as a round member with an opening 24 extending therethrough. It will be noted that the base 22 may take any shape suitable for the procedure, e.g. square. Additionally, the base 22 may be formed as a solid member that does not include the opening 24. The illustrated base 22 includes an outer rim 26 having a first circumference and an inner rim 28 having a second circumference that is less than the first circumference. Accordingly, the outer rim 26 and the inner rim 28 give the base 22 a ring shape.

A plurality of tines 30 extend from the base 22. It will be appreciated that the anchor 20 may include any number of tines 30. In an exemplary embodiment, the anchor 20 includes at least two tines 30 so that a tine 30 may be positioned on each side of the suture tape 10 during the procedure. In the illustrated embodiment, the tines 30 extend outward from the inner rim 28 of the base 22. In some embodiments, the tines 30 may extend outward from the outer rim 26 of the base 22 or from a location between the inner rim 28 and the outer rim 26. Each tine 30 includes a point 32 that is configured to embed the tine 30 into the patient's bone.

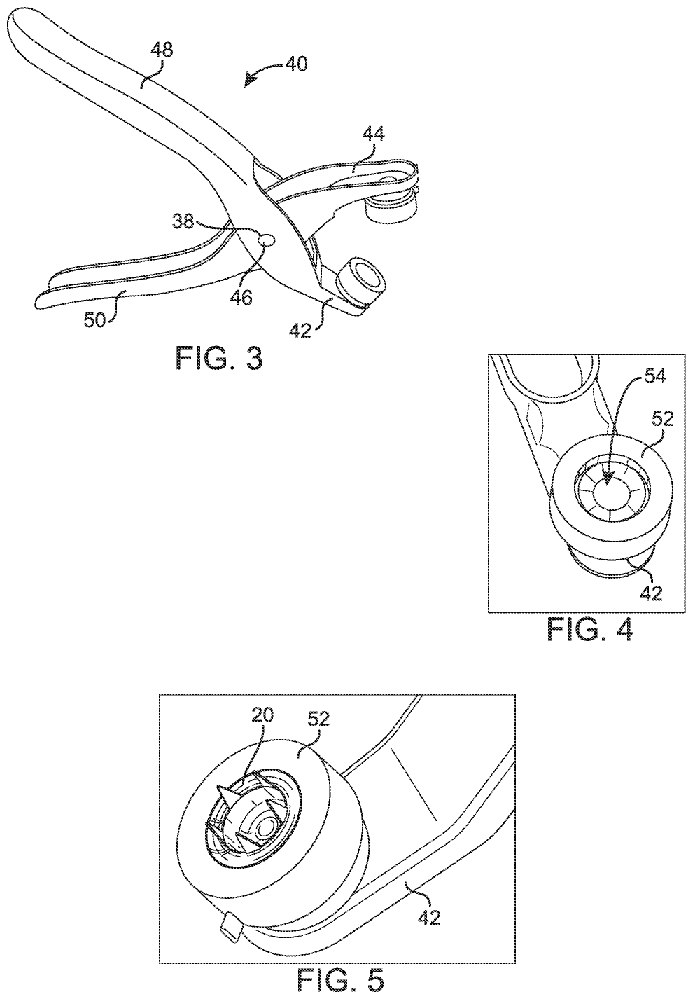

Referring now to FIG. 3 an instrument 40 is also included in the kit. In the exemplary embodiment, the instrument is a clamp. The instrument 40 includes a first arm 42 pivotally coupled to a second arm 44 at a pivot joint 46. A first grip 48 extends from the first arm 42, and a second grip 50 extends from the second arm 44. The first grip 48 and the second grip 50 are actuated, e.g. by a spring 38 position at the pivot joint 46, into a separated position so that the first arm 42 and second arm 44 are moved into an open position. By applying force to the first grip 48 and the second grip 50 to move the first grip 48 and the second grip 50 together, the first arm 42 and the second arm 44 are moved into a closed position, wherein the first arm 42 contacts the second arm 44. The force applied to the first grip 48 and the second grip 50 is transferred to the first arm 42 and the second arm 44.

As illustrated in FIG. 4 each arm 42, 44 includes a pad 52 that is utilized to retain an anchor 20, as illustrated in FIG. 5. The pad 52 is generally shaped to correspond to the shape of the anchor 20. In the illustrative embodiment, the pad 52 is generally circular and includes an opening 54. In some embodiments, the anchor 20 is removeably secured into the opening 54 during the procedure and prior to securing the anchor 20 to the patient's bone. The anchor 20 is held in a fixed position by the pad 52 so that the anchor 20 does not fall out of the instrument 40 during the procedure. In some embodiments, the pad 52 may be magnetic to retain a metallic anchor 20.

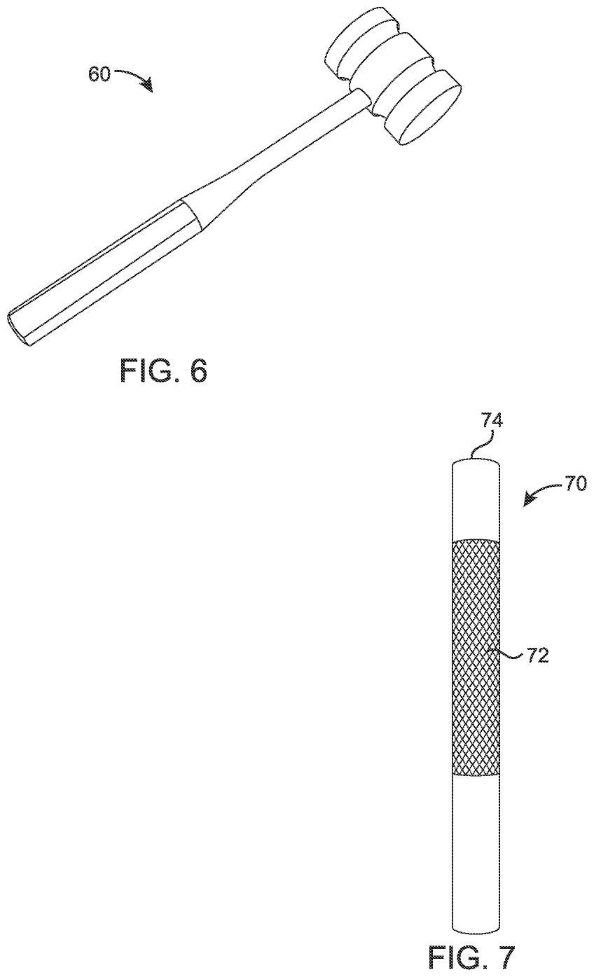

FIG. 6 illustrates another instrument 60 that is included in the kit. In some embodiments, the kit includes only one of the instrument 40 and the instrument 60. In other embodiments, the kit includes both the instrument 40 and the instrument 60 to provide flexibility to the surgeon when performing the procedure. In the illustrative embodiment, the instrument 60 is mallet that is configured to apply a striking force to a pin 70, as illustrated in FIG. 7. The pin 70 includes a solid body 72 having a striking end 74 that can withstand the force of the instrument 60. The pin 70 is configured to position against the anchor 20. A force is then applied to the pin 70 with the instrument 60 to secure the anchor 20 to the patient's bone.

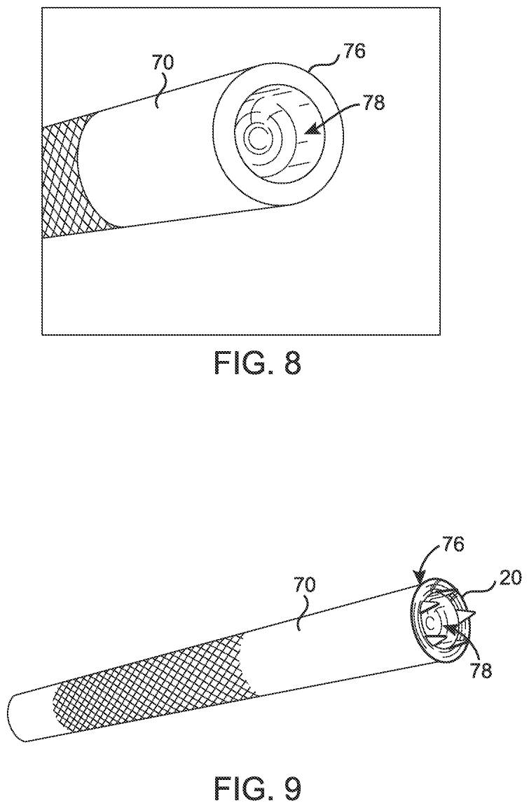

Referring to FIG. 8, an anchor end 76 of the pin 70 is generally circular, but may have any shape corresponding to the shape of the anchor 20. The end 76 includes a recess 78. In some embodiments, the anchor 20 is removeably secured in the recess 78 during the procedure and prior to securing the anchor 20 to the patient's bone, as shown in FIG. 9. The anchor 20 is held in a fixed position by the end 76 so that the anchor 20 does not fall out of the pin 70 during the procedure. In some embodiments, the end 76 may be magnetic to retain a metallic anchor 20.

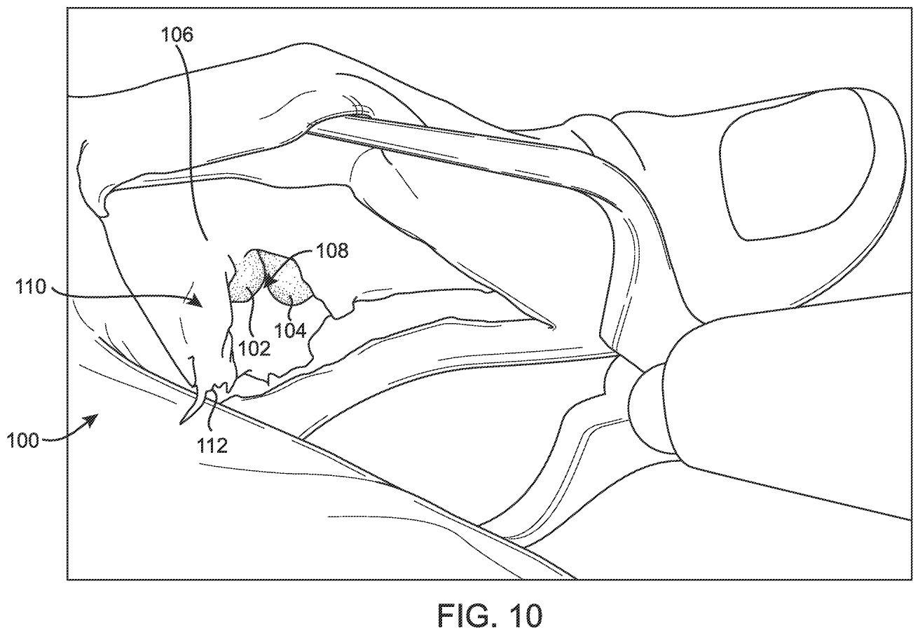

Referring now to FIG. 10, the illustrative hand 100 of the patient includes a proximal bone 102 and a distal bone 104. A ligament 106 typically extends between a joint 108 between the proximal bone 102 and the distal bone 104. Typically, the ligament 106 is attached to both the proximal bone 102 and the distal bone 104. In the illustrative embodiment, the ligament 106 is damaged. That is, the ligament 106 is attached at a fixed end 110 to the proximal bone 102 and detached at a free end 112 from the distal bone 104. It will be appreciated that the ligament 106 may be attached to the distal bone 104 and detached from the proximal bone 102. In some scenarios, the ligament 106 may be detached from both the proximal bone 102 and the distal bone 104.

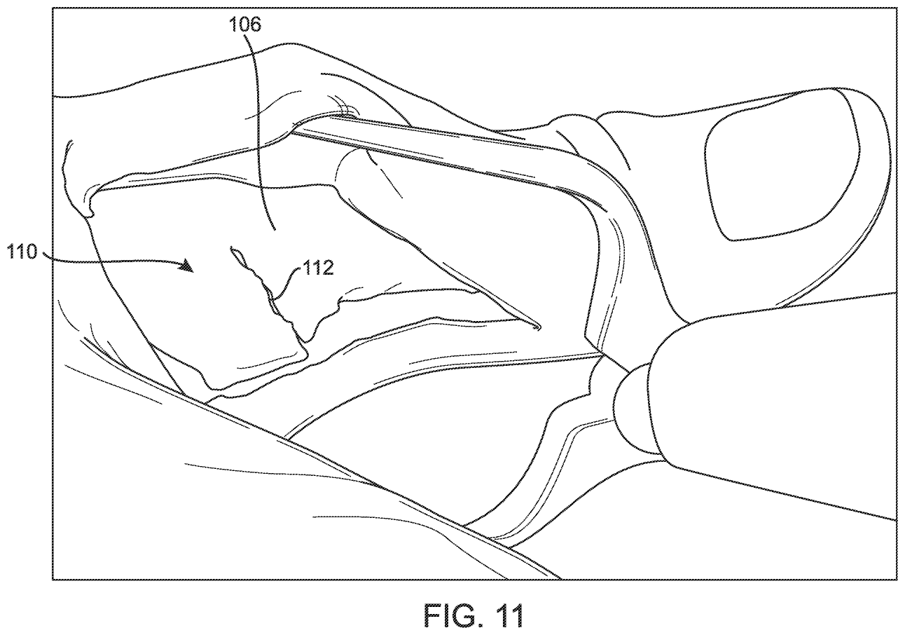

During a procedure to repair the ligament 106, the free end 112 of the ligament 106 is extended over the joint 108, as illustrated in FIG. 11. Generally, the free end 112 of the ligament 106 is extended over the joint 108 to a distance that achieves a predetermined tension in the ligament 106 post-surgery. The free end 112 of the ligament 106 is positioned on the distal bone 104. In this position, the ligament 106 will reattach to the distal bone 104 post-surgery.

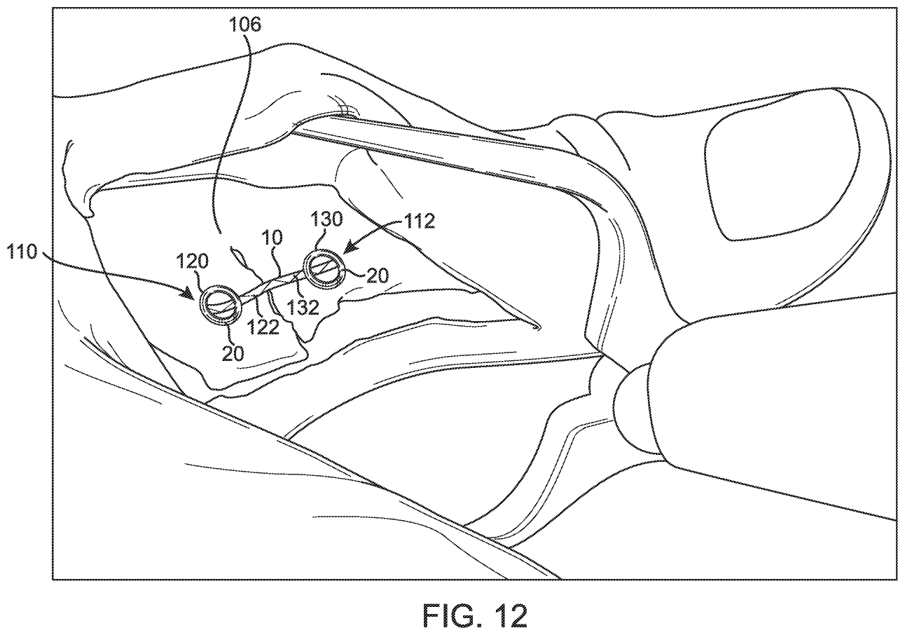

To secure the ligament 106 in place, the suture tape 10 is extended over the ligament 106, as illustrated in FIG. 12. That is, the suture tape 10 is extended over the ligament 106 from the fixed end 110 at the proximal bone 102 to the free end 112 at the distal bone 104. A pair of anchors 20 are positioned over the suture tape 10 at both the proximal bone 102 and the distal bone 104. It will be appreciated that any number of anchors 20 may be utilized to secure the suture tape 10.

More specifically, a proximal anchor 120 is positioned over the fixed end 110 of the ligament 106 at the proximal bone 102. The proximal anchor 120 is secured to the proximal bone 102 by pressing at least one tine 30 of the proximal anchor 120 into the proximal bone 102. In exemplary embodiment, at least one tine 30 is secured to a first side of the suture tape 10, and at least one tine 30 is secured to a second side of the suture tape 10. In another embodiment, each tine 30 of the plurality of tines 30 is pressed into the proximal bone 120. A first end 122 of the suture tape 10 and the fixed end 110 of the ligament 106 are secured between the proximal bone 102 and the proximal anchor 120.

A distal anchor 130 is positioned over the free end 112 of the ligament 106 at the distal bone 104. The distal anchor 130 is secured to the distal bone 104 by pressing at least one tine 30 of the distal anchor 130 into the distal bone 104. In exemplary embodiment, at least one tine 30 is secured to a first side of the suture tape 10, and at least one tine 30 is secured to a second side of the suture tape 10. In another embodiment, each tine 30 of the plurality of tines 30 is pressed into the distal bone 120. A second end 132 of the suture tape 10 and the free end 112 of the ligament 106 are secured between the distal bone 104 and the distal anchor 130.

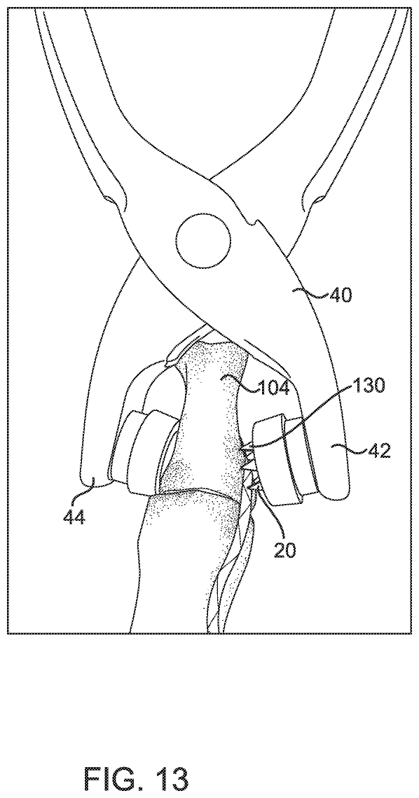

Referring to FIG. 13 the instrument 40 may be utilized to secure the anchors 20 to the respective bones. For example, the distal anchor 130 is secured to the distal bone 104 by squeezing the distal anchor 130 with the instrument 40. In such an embodiment, pressure is applied to the distal anchor 130 with the first arm 42 of the instrument 40, wherein the distal anchor 130 is secured to the first arm 42, and the pressure is likewise applied to the distal bone 104 with the second arm 44 of the instrument 40.

Likewise, the proximal anchor 120 is secured to the proximal bone 102 by squeezing the proximal anchor 120 with the instrument 40. In such an embodiment, pressure is applied to the proximal anchor 120 with the first arm 42 of the instrument 40, wherein the proximal anchor 120 is secured to the first arm 42, and the pressure is likewise applied to the proximal bone 102 with the second arm 44 of the instrument 40.

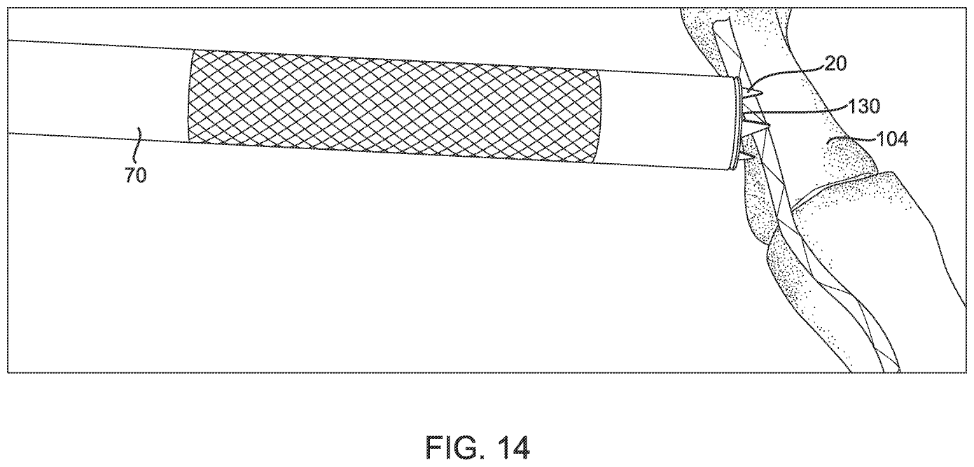

Referring now to FIG. 14, the instrument 60 and the pin 70 may be utilized to secure the anchors 20 to the respective bones. For example, the distal anchor 130 is secured to the distal bone 104 by applying force to the distal anchor 130 by striking the pin 70 with the instrument 60, wherein the distal anchor 130 is secured to the pin 70. Likewise, the proximal anchor 120 is secured to the proximal bone 102 by applying force to the proximal anchor 120 by striking the pin 70 with the instrument 60, wherein the proximal anchor 120 is secured to the pin 70.

Accordingly, the ligament 106 is repaired without drilling into either the proximal bone 102 or the distal bone 104. After the procedure, the surgical site is closed using standard methods of suturing a surgical site. The surgical site is closed with the suture tape 10, the proximal anchor 120, and the distal anchor 130 positioned within the surgical site. Over time, the suture tape 10 and the anchors 20 retain the ligament in position against the distal bone 104 allowing the ligament 106 to reattach to the distal bone 104.

Any theory, mechanism of operation, proof, or finding stated herein is meant to further enhance understanding of principles of the present disclosure and is not intended to make the present disclosure in any way dependent upon such theory, mechanism of operation, illustrative embodiment, proof, or finding. It should be understood that while the use of the word preferable, preferably or preferred in the description above indicates that the feature so described can be more desirable, it nonetheless cannot be necessary and embodiments lacking the same can be contemplated as within the scope of the disclosure, that scope being defined by the claims that follow.

In reading the claims it is intended that when words such as “a,” “an,” “at least one,” “at least a portion” are used there is no intention to limit the claim to only one item unless specifically stated to the contrary in the claim. When the language “at least a portion” and/or “a portion” is used the item can include a portion and/or the entire item unless specifically stated to the contrary.

It should be understood that only selected embodiments have been shown and described and that all possible alternatives, modifications, aspects, combinations, principles, variations, and equivalents that come within the spirit of the disclosure as defined herein or by any of the following claims are desired to be protected. While embodiments of the disclosure have been illustrated and described in detail in the drawings and foregoing description, the same are to be considered as illustrative and not intended to be exhaustive or to limit the disclosure to the precise forms disclosed. Additional alternatives, modifications and variations can be apparent to those skilled in the art. Also, while multiple inventive aspects and principles can have been presented, they need not be utilized in combination, and many combinations of aspects and principles are possible in light of the various embodiments provided above.

1. A method of repairing a ligament, wherein the ligament includes a fixed end fixed to a first bone and a free end disconnected from an adjacent second bone, the method comprising:
extending the free end of the ligament over a joint between the first bone and the second bone,
positioning the free end of the ligament on the second bone,
positioning suture tape across the ligament from the fixed end of the ligament to the free end of the ligament,
positioning a first anchor over the fixed end of the ligament,
securing the first anchor to the first bone by pressing a tine of the first anchor into the first bone, wherein a first end of the suture tape and the fixed end of the ligament are secured between the first bone and the first anchor,
positioning a second anchor over the free end of the ligament, and
securing the second anchor to the second bone by pressing a tine of the second anchor into the second bone, wherein a second end of the suture tape and the free end of the ligament are secured between the second bone and the second anchor.

2. The method of claim 1, further comprising:
securing the first anchor to the first bone by squeezing the first anchor with a clamp, and
securing the second anchor to the second bone by squeezing the second anchor with the clamp.

3. The method of claim 2, further comprising:
applying a pressure to the respective anchor with a first arm of the clamp, and
applying the pressure to the respective bone with a second arm of the clamp.

4. The method of claim 1, further comprising:
securing the first anchor to the first bone by applying force to the first anchor with a mallet, and
securing the second anchor to the second bone by applying force to the second anchor with the mallet.

5. The method of claim 4, further comprising:
removeably securing each respective anchor to an end of a pin, and
applying the respective force to the pin with the mallet.

6. The method of claim 1, further comprising extending the free end of the ligament over the joint to a distance that achieves a predetermined tension in the ligament.

7. The method of claim 1, wherein:
each respective anchor includes a plurality of tines, and
the method further includes pressing each of the plurality of tines into the respective bone.

8. The method of claim 1, wherein:
the ligament is repaired at a surgical site, and
the method further includes closing the surgical site with the suture tape, the first anchor, and the second anchor positioned within the surgical site.

9. The method of claim 1, wherein positioning the suture tape across the ligament further comprises positioning a suture tape that includes an absorbable material.

10. The method of claim 1, further comprising repairing the ligament without drilling into either the first bone or the second bone.

11. A surgical kit for repairing a ligament, wherein the ligament includes a fixed end fixed to a first bone and a free end disconnected from an adjacent second bone, the kit comprising:
suture tape configured to be positioned across the ligament from the fixed end of the ligament to the free end of the ligament,
a first anchor configured to position over the fixed end of the ligament,
a second anchor configured to be positioned over the free end of the ligament, and
an instrument configured to secure the first anchor to the first bone by pressing a tine of the first anchor into the first bone, wherein a first end of the suture tape and the fixed end of the ligament are configured to be secured between the first bone and the first anchor, wherein the instrument is further configured to secure the second anchor to the second bone by pressing a tine of the second anchor into the second bone, wherein a second end of the suture tape and the free end of the ligament are configured to be secured between the second bone and the second anchor.

12. The surgical kit of claim 11, wherein the instrument includes a clamp.

13. The surgical kit of claim 12, wherein the clamp includes:
a first arm configured to apply pressure to the respective anchor, and
a second arm configured to apply pressure to the respective bone.

14. The surgical kit of claim 13, wherein each respective anchor is removeably secured to the first arm of the clamp.

15. The surgical kit of claim 11, wherein the instrument includes a mallet.

16. The surgical kit of claim 15, further comprising a pin configured to position against the respective anchor, wherein force is applied to the pin with the mallet.

17. The surgical kit of claim 16, wherein each respective anchor is removeably secured to the pin.

18. The surgical kit of claim 11, wherein each respective anchor includes a plurality of tines.

19. The surgical kit of claim 11, wherein each respective anchor includes:
a first tine configured to position on a first side of the suture tape, and
a second tine configured to position on a second side of the suture tape.

20. The surgical kit of claim 11, wherein the suture tape includes an absorbable material.
